(19) United States
(12) Patent Application Publication
(10) Pub. No.: US 2024/0166659 A1
(43) Pub. Date: May 23, 2024

(54) A PORPHYRIN COMPOSITION AND PROCESS OF PRODUCING THE PORPHYRIN COMPOSITION

(71) Applicant: Novomer, Inc., Rochester, NY (US)

(72) Inventor: Alison M. Wilders, Rochester, NY (US)

(21) Appl. No.: 18/552,683

(22) PCT Filed: Mar. 10, 2022
(86) PCT No.: PCT/US2022/019775

§ 371 Date: Sep. 27, 2023

Related U.S. Application Data
(60) Provisional application No. 63/174,581, filed on Apr. 14, 2021.
(60) Provisional application No. 63/171,152, filed on Apr. 6, 2021.

(51) Int. Cl. C07D 487/22; B01J 31/02

(52) U.S. Cl. CPC C07D 487/22; B01J 31/0232

(57) ABSTRACT

A method comprising: (a) combining a solvent, salt, pyrrole or substituted pyrrole, and an aliphatic or aromatic aldehyde in a vessel; (b) charging the vessel with a catalyst, a salt, and a solvent to form a porphyrinogen of the pyrrole or substituted pyrrole; and (c) oxidizing the formed porphyrinogen of the pyrrole or substituted pyrrole with an oxidizing agent to form a porphyrin; wherein the porphyrin comprises a residue of the pyrrole or substituted pyrrole having the aliphatic or aromatic groups derived from the aliphatic or aromatic aldehyde pendant from the heterocycle.

### CROSS-REFERENCE TO RELATED APPLICATION(S)

This application claims priority to of U.S. Provisional Patent Application Nos. 63/171,152, filed on Apr. 6, 2021, and 63/174,581, filed on Apr. 14, 2021 the entire disclosure of which are hereby incorporated by reference.

### FIELD

This disclosure relates to a porphyrin composition that is used in the production of lactones and more specifically a tetraphenylporphyrin that is used to form a carbonylation catalyst that is used to produce beta propiolactone.

### BACKGROUND

When making lactones, a carbonylation catalyst is typically used to facilitate a reaction from materials such as carbon monoxide and an epoxide to produce the lactones. The carbonylation catalyst can be formed from a porphyrin and specifically tetraphenylporphyrin that is as a precursor to the carbonylation catalyst. There are many different carbonylation catalysts that may be used in the carbonylation process; however, many of these carbonylation catalysts are cost prohibitive and; thus may not be compatible in producing low cost lactones. Further the processes for forming porphyrins may require harsh, acidic conditions, have tar as a byproduct, use highly toxic or corrosive materials, may not be scalable to a commercial process, or a combination thereof. Some examples of methods to form tetraphenylporphyrins are available in EP Patent No. 0308791; A Simplified Synthesis for meso-Tetraphenylporphin, Vol. 32, p 476, 1966 by Alan D. Alder; Rothemund and Adler-Longo Reactions Revisited: Synthesis of Tetraphenylporphyrins under Equilibrium Conditions; J. Org. Chem. 1987, 52, 827-836 by Jonathan S. Lindsey et al.; Beneficial Effects of Salts on an Acid-Catalyzed Condensation Leading to Porphyrin Formation Tetrahedron, Vol. 53, No 37, pp12339-12360, 1997, by Feirong Li et al.; and A survey of Acid Catalysts for Use in Two-Step, One-Flask Syntheses of Meso-Substituted Porphyrinic Macrocycles, American Chemical Society Org. Lett., Vol. 2, No. 12, 2000, by G. Richard Geier III et al. the teachings of which are expressly incorporated by reference herein for all purposes. Many of the current laboratory schemes may not be adaptable to production scale and being commercially viable because these processes may require harsh, acidic conditions, produce large amounts of tar which can complicate purification, or produce stoichiometric amounts of hydroquinone byproducts that are difficult to remove without column chromatography.

What is needed are methods to produce a carbonylation catalyst from novel components. What is needed are methods to produce a high yield carbonylation catalyst. It is desirable to produce a carbonylation catalyst in real time that is fed into a process for producing lactones. What is needed is a method of synthesizing a porphyrin with hydrogen peroxide and preferably in an absence of acetic acid. It would be desirable to produce a carbonylation catalyst from components that do not result in large amounts of tar, use large amounts of corrosive materials produce stoichiometric amounts of hydroquinone byproducts that are difficult to remove without column chromatography, or a combination thereof. What is needed is a method of synthesizing a porphyrin that is scalable to a commercial process.

### SUMMARY

The present teachings provide: a method comprising: (a) combining a solvent, salt, pyrrole or substituted pyrrole, and an aliphatic or aromatic aldehyde in a vessel; (b) charging the vessel with a catalyst, a salt, and a solvent to form a porphyrinogen of the pyrrole or substituted pyrrole; and (c) oxidizing the formed porphyrinogen of the pyrrole or substituted pyrrole with an oxidizing agent to form a porphyrin; wherein the porphyrin comprises a residue of the pyrrole or substituted pyrrole having the aliphatic or aromatic groups derived from the aliphatic or aromatic aldehyde pendant from the heterocycle.

The present teachings provide methods to produce a carbonylation catalyst from novel components. The present teachings provide methods to produce a high yield carbonylation catalyst. The present teachings provide a carbonylation catalyst that is produced in real time that is fed into a process for producing lactones. The present teachings provide a method of synthesizing a porphyrin with hydrogen peroxide and preferably in an absence of acetic acid. The present teachings a carbonylation catalyst from components that do not result in large amounts of tar, use large amounts of corrosive materials produce stoichiometric amounts of hydroquinone byproducts that are difficult to remove without column chromatography, or a combination thereof. The present teachings provide a method of synthesizing a porphyrin that is scalable to a commercial process.

### BRIEF DESCRIPTION OF THE DRAWING

FIG. 1 is a flow chart of a process of producing a porphyrin.

FIG. 2 is a flow chart of metalating a porphyrin to form a carbonylation catalyst.

FIG. 3 is a flow chart of forming a lactone with a carbonylation catalyst and converting the lactone.

### DETAILED DESCRIPTION

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

One or more as used herein means that at least one, or more than one, of the recited components may be used as disclosed. Residue with respect to an ingredient or reactant used to prepare the polymers or structures disclosed herein means that portion of the ingredient that remains in the polymers or structures after inclusion as a result of the methods disclosed herein. Substantially all as used herein means that greater than 90 percent of the referenced parameter, composition, structure or compound meet the defined criteria, greater than 95 percent, greater than 99 percent of the referenced parameter, composition or compound meet the defined criteria, or greater than 99.5 percent of the referenced parameter, composition or compound meet the defined criteria. Portion as used herein means less than the full amount or quantity of the component in the composition, stream, or both. Precipitate as used herein means a solid compound in a slurry or blend of liquid and solid compounds. Phase as used herein means a solid precipitate or a liquid or gaseous distinct and homogeneous state of a system with no visible boundary separating the phase into parts. Parts per weight means parts of a component relative to the total weight of the overall composition. A catalyst component as used herein means a metal centered compound, a metal carbonyl, a Lewis acid, a Lewis acid derivative, a metal carbonyl derivative, or any combination thereof. A catalyst as used herein includes at least a cationic compound and an anionic compound. An organic compound as used herein includes any compound that is free of a metal atom. An inorganic compound as used herein includes compounds that include at least one metal atom. Composition as used herein includes all components in a stream, reactant stream, product stream, slurry, precipitate, liquid, solid, gas, or any combination thereof that are containable within a single vessel.

The present teachings disclose a method of producing a carbonylation catalyst. The carbonylation catalyst functions to convert a precursor material, convert materials into a catalyst, form a portion of a catalyst, or a combination thereof. The carbonylation catalyst is produced from a porphyrin. The method herein is directed to a ligand precursor (e.g., a porphyrin) of the carbonylation catalyst. The ligand precursor may be produced in real time from precursor materials in real time, continuously, as a batch, or a combination thereof. The precursor materials may be a combination of two or more, three or more, four or more, or even five or more materials.

The method may begin with two or more precursor materials. The precursor materials may be a pyrrole or substitute pyrrole and an aliphatic aldehyde or an aromatic aldehyde, or a combination thereof. The precursor materials may include material that is electron-donating or electron-withdrawing. The pyrrole and the aldehyde may be combined into a vessel. The vessel may be a dried vessel. The vessel may be free of water. The pyrrole may be freshly distilled before being added to the vessel. A catalyst, a salt, or both may be added to the two or more mixed precursor materials. The catalyst, the salt, or both may be added with a solvent. The catalyst, the salt, and the solvent may be added simultaneously, in series, intermittently, over a period of time, or a combination thereof. The two or more precursor materials, salt, catalyst, solvent, or a combination thereof may be mixed in the vessel. The mixing may be performed for a predetermined amount of time. The predetermined amount of time may be about 1 min or more, about 3 min or more, or about 5 min or more. The predetermined amount of time may be about 12 hours or less, about 6 hours or less, about 1 hour or less, about 30 min or less, or about 15 min or less. The predetermined amount of time may be a sufficient amount of time so that all of the pyrrole and all of the aldehyde are consumed, bonded, or both. The predetermined amount of time may be a sufficient amount of time so that substantially all of the pyrrole and all of the aldehyde are consumed in a condensation (e.g., a reaction in which the aldehyde and pyrrole reaction leading to water as a byproduct); converted into an porphyrinogen; or both (e.g., 90 percent or more, 95 percent or more, or 99 percent or more are consumed or converted).

The porphyrinogen may be maintained in the vessel or moved to a second vessel. The porphyrinogen may be contacted by an oxidizing agent. The oxidizing agent may remove hydrogen, remove electrons, or both. The oxidizing agent may convert the porphyrinogen to a porphyrin. The porphyrinogen and the oxidizing agent may be mixed for a second predetermined amount of time. The second predetermined amount of time may be about 6 hours or more, 8 hours or more, or 12 hours or more. The second predetermined amount of time may be about 1 week or less, 5 days or less, 3 days or less, 1 day or less, or about 15 hours or less. The second predetermined amount of time may be long enough that six hydrogens are removed from the porphyrinogen and double bonds are formed in their place. The second predetermined amount of time may be a sufficient amount of time so that a porphyrin is produced.

The porphyrin may be maintained within the vessel. The porphyrin may be moved to a catalyst synthesis system or vessel. The porphyrin may be contacted with a metalating agent. The metalating agent may release a metal atom that is received within the porphyrin. The metalating agent may be any of the compositions discussed herein. The metalating agent may form a metalated porphyrin. The metalating porphyrin may be maintained within the first vessel. The metalated porphyrin may be moved from a first vessel to a second vessel.

The metalated porphyrin may be contacted with one or more metal carbonyls. Preferably, the metalated porphyrin is contacted with a single metal carbonyl. The metalated porphyrin may be reacted with a metal carbonyl to produce a carbonylation catalyst.

The carbonylation catalyst formation step may include contacting the Lewis acids containing the halogen or the alkyl group with a polar ligand, a metal carbonyl additive, or both to from the carbonylation catalyst. The Lewis acid containing the halogen or alkyl group may be added in a molar ratio of about 1:1. The metal carbonyl additive may contain at least a metal carbonyl that is anionic and a cationic group that is configured to cleave and bond with the alkyl group or the halogen of the metal centered compound. The cationic group may be one or more of an alkali metal, (R4)₃Si—, any counterion sufficient to ionically bond and/or balance the metal carbonyl, or any combination thereof, where R4 is independently selected from a phenyl, halophenyl, hydrogen, alkyl, alkylhalo, alkoxy, or any combination thereof. In examples where the metal carbonyl additive cleaves or decouples the alkyl group, the alkyl group may couple with the cationic group, and the alkyl group and cationic group could be removed via any filtration or removal means described herein. In examples where the metal carbonyl additive cleaves the halogen from the metal centered compound and is contacted with the polar compound, the halogen bonds with the cationic group of the metal carbonyl additive and the Lewis acid containing the polar compound is formed. Any byproducts can be removed by any other removal or separation steps described herein. After the metal carbonyl additive cleaves or decouples the alkyl group, the Lewis acid may combine with the polar ligand to form a cationic species. The Lewis acid containing the polar ligand then contacts with the anionic metal carbonyl of the metal carbonyl additive and forms the regenerated carbonylation catalyst.

The steps to form the carbonylation catalyst may be performed under conditions that are moisture and oxygen free. For example, the catalyst formation steps may be performed within a dry glove box, on a Schlenk line, or in a reactor under an inert atmosphere (i.e., nitrogen). The catalyst formation steps may be performed under a nitrogen, argon, or any other inert gas. During the catalyst formation steps, the Lewis acid, the polar ligand, the metal carbonyl, or any combination thereof may be contacted and agitated by stirring for a period of time sufficient to form the carbonylation catalyst. The period of time for stirring the components may be about 5 minutes or more, about 30 minutes or more, about 60 minutes or more. The period of time for stirring the components may be about 24 hours or less, about 12 hours or less, or about 6 hours or less. The components in the catalyst formation steps may be completed under ambient temperature and/or pressure. Additional steps to make the regenerated catalyst can be found in U.S. Pat. No. 6,852,865B2 and U.S. Pat. No. 8,481,75661, both of which are included herein by reference in their entirety.

The metal carbonyl of the carbonylation catalyst functions to provide the anionic component of the carbonylation catalyst. The carbonylation catalyst may include one or more, two more, or a mixture of metal carbonyls. The metal carbonyl may be capable of ring-opening an epoxide and facilitating the insertion of CO into the resulting metal carbon bond. In some examples, the metal carbonyl may include an anionic metal carbonyl moiety. In other examples, the metal carbonyl compound may include a neutral metal carbonyl compound. The metal carbonyl may include a metal carbonyl hydride or a hydrido metal carbonyl compound. The metal carbonyl may be a pre-catalyst which reacts in situ with one or more reaction components to provide an active species different from the compound initially provided. The metal carbonyl includes an anionic metal carbonyl species. in some examples, the metal carbonyl may have the general formula [Q_{d}M′e(CO)_{w}]^{y+}, where Q is an optional ligand, M′ is a metal atom, d is an integer between 0 and 8 inclusive, e is an integer between 1 and 6 inclusive, w is a number such as to provide the stable anionic metal carbonyl complex, and y is the charge of the anionic metal carbonyl species. The metal carbonyl may include monoanionic carbonyl complexes of metals from groups 5, 7 or 9 of the periodic table or dianionic carbonyl complexes of metals from groups 4 or 8 of the periodic table. The metal carbonyl may contain cobalt, manganese, ruthenium, or rhodium. Exemplary metal carbonyls may include [Co(CO)_{4l ]}⁻, [Ti(CO)e]²⁻, [V(CO)_{6]}⁻, [Rh(CO)⁴]⁻, [Fe(CO)₄]²⁻, [Ru(CO)₄]2−, [Os(CO)₄]²⁻, [Cr₂(CO)₁₀]²⁻, [Fe₂(CO)₈]²⁻, [Tc(CO)₅]⁻, [Re(CO)₅]⁻, and [Mn(CO)₅]⁻. The metal carbonyl may be a mixture of two or more anionic metal carbonyl complexes in the carbonylation catalysts used in the methods.

The Lewis acid or Bronsted acid catalyst may function to catalyze a condensation reaction. The Lewis acid or Bronsted acid catalyst may be one or more of formic acid, acetic acid, aluminum chloride, or any combination thereof.

The metal alkyl compound may function to coordinate a metal in one or more ligands to form a Lewis acid containing a halogen or an alkyl group. The metal alkyl compound may be any compound containing a metal and/or one or more alkyl groups and/or halogen group. The metal of the metal alkyl compound may be one or more of aluminum, chromium, or any combination thereof. The meal alkyl compound may include one or more of CrCl₂, (Et)₂ AlCl or (Et)₃Al, or any combination thereof.

A metal carbonyl additive functions to deliver a metal carbonyl to a Lewis acid that is suitable to combine and form the carbonylation catalyst. The metal carbonyl additive may function to decouple a halogen or a alkyl group from a Lewis acid to form the carbonylation catalyst that includes the Lewis acid and metal carbonyl combination. The metal carbonyl additive includes at least a metal carbonyl as described herein and a cationic compound. The cationic compound may include lithium, sodium, potassium, rubidium, cesium, beryllium, magnesium, calcium, strontium, barium, radium, or any combination thereof. The metal carbonyl additive may be a salt. The metal carbonyl additive may be a silicon salt in the form of R₃Si—, where R is independently selected from a phenyl, halophenyl, hydrogen, alkyl, alkylhalo, alkoxy, or any combination thereof. The metal carbonyl additive may be NaCo(CO)₄, Co₂(CO)₈, HCo(CO)₄, or any combination thereof. Where a Lewis acid containing a halogen is formed after the metalation step, NaCo(CO)₄ may be used to form the carbonylation catalyst. Where a Lewis acid containing an alkyl group is formed, Co₂(CO)₈ or HCo(CO)₄ may be used to form the carbonylation catalyst.

The precursor materials function to produce a ligand precursor and preferably a porphyrin ligand. The precursor materials (e.g., a porphyrin) to form a carbonylation catalyst may be formed in a macrocycle system, a macrocycle process, or both. The precursor materials may combine together in a first vessel and then be added to a second vessel. The precursor materials may be added to a reaction vessel. The precursor materials be added together in a method taught herein. The precursor materials may include a pyrrole, a substituted pyrrole, an aldehyde, a catalyst, a salt, a solvent, an oxidizing agent, a metalating agent, a metal carbonyl, or a combination thereof.

The porphyrin system functions to combine a plurality of precursor materials together to form a carbonylation catalyst. The porphyrin system may be one or more vessels. The porphyrin system may be a batch system, a continuous system, or a semi-batch. The porphyrin system may be or include a reaction vessel, a mixing vessel, a filter, a vacuum, a condenser, a distillation column, or a combination thereof. The reaction vessel may receive all of the precursor materials. The reaction vessel may hold a sufficient amount of material to form all of the porphyrin needed for the process. The reaction vessel may include heating, mixing, filtration, cooling, or a combination thereof. The reaction vessel may be maintained at ambient temperature. The reaction vessel may include vacuum. The reaction vessel may be maintained at ambient pressure, negative pressure, positive pressure, or alternated therebetween. The reaction vessel may be maintained at an ambient temperature and ambient pressure for all or a portion of the process. The reaction vessel may include an inlet filter or membrane, an outlet filter or membrane, or an internal membrane or filter. The reaction vessel may filter when passing the components from one vessel to another vessel. The reaction vessel may filter the components before components are concentrated, slurried, finalized, or a combination thereof. The porphyrin system may be a liquid system, a semi-solid system, a dry system, or a combination thereof. The porphyrin system may receive liquid components and output a solid, a crystalline substance, or both. The porphyrin system may receive an initial charge of a pyrrole and an aldehyde.

The pyrrole functions as one of the precursor materials that produces a porphyrin or preferably tetraphenylporphyrin. The pyrrole may be a single ring. The pyrrole may be a substituted pyrrole. The pyrrole may be mono-substituted or bi-substituted. The pyrrole may include 4 or more carbon atoms. The pyrrole may include 4 or more carbon atoms in a ring. The pyrrole may have a chemical formula of C₄H ₅ N. The pyrrole may have the following formula:

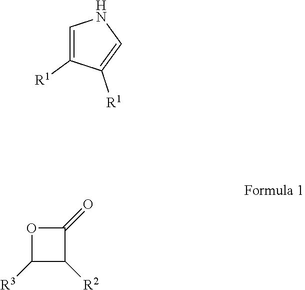

Wherein R¹ may be separately in each occurrence a hydrogen, halogen, alkyl group, aryl or substituted aryl, ester, ketone, alkyne, alkene, nitrate, sulfate, or a combination thereof. The pyrrole may be added in a sufficient amount so that all of the pyrrole is consumed, all of the aldehyde is consumed, or both. The pyrrole may be in an amount 0.25 equivalent or more, 0.5 equivalent or more, 0.75 or more, 1 or more of each initial component (e.g., the pyrrole and aldehyde). The pyrrole may be added in a molar ratio relative to the aldehyde. The molar ratio of pyrrole to aldehyde may be a molar ratio of about 0.5:1 or more, about 0.75:1 or more, about 1:1 or more, about 1:0.75 or less, about 1:0.5 or less. Preferably, the pyrrole and aldehyde may be added in equal amounts.

The aldehyde may be an aliphatic aldehyde or an aromatic aldehyde. The aldehyde may function to bond to the pyrrole to create a macrocycle ring. The aldehyde may bond two pyrroles together. The aldehydes may bond in an alternating fashion with the pyrroles. The aldehydes may be represented by the following formula:

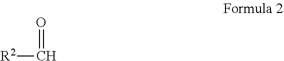

wherein R² is separately in each occurrence an alkene, ester, ketone, haloalkane, alkyne, a carbocycle, furyl, aryl, substituted aryl, naphthyl, anthracenyl, or a combination thereof. R² at each occurrence is independently hydrogen, halogen, —OR², —NR^{y}₂, —SR^{y}, —CN, —NO₂, —SO₂R^{y}, —SOR^{y}, —SO₂NR^{y}₂; —CNO, —NR^{y}SO₂R^{y}, —NCO, —N₃, —SiR^{y}₃; Or an optionally substituted group selected from the group consisting of C1-20 aliphatic; C1-20 heteroaliphatic having 1-4 heteroatoms independently selected from the group consisting of nitrogen, oxygen, and sulfur; 6- to 10-membered aryl; 5- to 10-membered heteroaryl having 1-4 heteroatoms independently selected from nitrogen, oxygen, and sulfur; and 4- to 7-membered heterocyclic having 1-2 heteroatoms independently selected from the group consisting of nitrogen, oxygen, and sulfur, where two or more Regroups may be taken together to form one or more optionally substituted rings. Preferably, R 2 may be phenyl or a substituted aryl group. The aldehyde may be added in a sufficient amount so that all of the pyrrole is consumed, all of the aldehyde is consumed, or both. The aldehyde may be in an amount 0.25 equivalent or more, 0.5 equivalent or more, 0.75 or more, 1 or more of each initial component (e.g., the pyrrole and aldehyde). The pyrrole may be added in a ratio relative to the aldehyde.

The catalyst functions to activate the pyrrole, the aldehyde, or both. The catalyst functions to cause the pyrrole and the aldehyde to react. The catalyst may function to combine the pyrrole and the aldehyde at room temperature. The catalyst may cause substantially all of the pyrrole, the aldehyde, or both to combine or to be consumed. The catalyst may be a Lewis acid or a Bronstead acid. The catalyst may be one or more of selected from the group consisting of BF₃-etherate, TFA, p-CH₃C₆H₄SO₃H·H₂O, CH₃SO₃H, SbF₅, GeBr₄, PBr₅, BBr₃, TiBr₄, CCl₃CO₂H, CuCl₂, AlCl₃, MgBr₂, TiCl₄, GaCl₂, SnCl₄, FeCl₃, HCl, SiCl₄, BCl₃, TeCl₄, C₆F₅CO₂H, Gel₄, BEt₃. Preferably, the catalyst is one or more selected from the group consisting of BF₃-etherate, TFA, p-CH₃C₆H₄SO₃H·H₂O, CH₃SO₃H, SbF₅, GeBr₄, PBr₅, BBr₃, TiBr₄, CCl₃CO₂H, CuCl₂, AlCl₃, MgBr₂, TiCl₄, GaCl₂, SnCl₄, FeCl₃, HCl, SiCl₄, BCl₃, or TeCl₄. More preferably, the catalyst is one or more selected from the group consisting of BF₃-etherate, TFA, or p-CH₃C₆H₄SO₃H·H₂O. The catalyst may be a cationic exchange catalyst. The catalyst may be an acid functionalized zeolite. The catalyst may be boron trifluoride etherate. The catalyst may be present in an amount of about 1 mol % or more, about 3 mol % or more, about 5 mol % or more, about 7 mol % or more or about 10 mol % or more based on the moles of the pyrrole. The catalyst may be present in an amount of about 50 mol % or less, about 40 mol % or less; about 30 mol % or less; about 20 mol % or less; or about 15 mol % or less based on the moles of the pyrrole. The catalyst may be added at a same time as a salt, after a salt, before a salt, or a combination thereof.

The salt may assist in reacting the pyrrole and the aldehyde. The salt may increase a rate of reaction at room temperature. The salt may increase a rate of reaction by about 10 percent or more, about 20 percent or more, about 30 percent or more, even about 40 percent or more, or about 60 percent or less relative to a reaction with no salt. The salt and catalyst may work together to react the pyrrole and the aldehyde. The salt may be one or more of the salts selected from the group consisting of LiCl, NaCl, KCl, CsCl, MgCl₂, CaCl₂, NH₄Cl, Me₄NCl, NaBPh₄, BaBr, Kl, Na₂SO₄, MgSO₄, CaSO₄, NaBF₄, Me₄NBF₄, or KF. The salt may be one or more of the salts selected from the group consisting of LiCl, NaCl, KCl, CsCl, MgCl₂, CaCl₂, Paraquat 2Cl—, NH₄Cl, Me₄NCl, NaBPh₄, BaBr, or Kl. The salt is one or more of the salts selected from the group consisting of NaCl, Me4NCl, or NaBPh4. The salt may be a metal salt. The salt may be a metal salt with an anion selected from the group consisting of Cl—, Ph4B—, Br—, or l—. Preferably, the salt is sodium chloride. The salt may be added in an amount of about 0.25 M or more, 0.1 mM or more, about 1 mM or more, or about 10 mM or more. The salt may be added in an amount of about 1000 mM or less, about 500 mM or less, about 250 mM or less, or about 100 mM or less. The salt may be added with a solvent, before a solvent, after a solvent, or a combination thereof.

The solvent functions to create a solution so that the pyrrole and the aldehyde may be mixed together, suspended, combined, reacted, or a combination thereof. The solvent may be a non-reactive substance that may suspend the precursor materials so that the precursor materials may react. The solvent may be added in a sufficient concentration so that the precursor materials (e.g., pyrrole or aldehyde) may be suspended, mixed, reacted, or both. The solvent may be a halogenated solvent. The solvent may have a boiling point above ambient temperatures (e.g., about 20±2° C.). The solvent may be free of water or may not be water. The solvent may be a halogenated hydrocarbon. The solvent may be dichloromethane or chloroform. The solvent may be present in an amount of about 0.001 M or more, about 0.01 M or more, about 0.05 M or more, or about 0.1 M or more of the total composition. The solvent may be present in an amount of about 0.5 M or less, about 0.4 M or less, about 0.3 M or less, about 0.2 M or less or about 0.15 M or less (e.g., ±0.05 M). The precursor materials when reacted form a porphyrinogen. The condensation may be performed at any temperature at which the pyrrole and aldehyde condense. The condensation may be performed at ambient temperatures. The condensation may be performed at a temperature of greater than 0° C., at about 10° C. or greater or about 20° C. or greater. The condensation may be performed at a temperature of about 30° C. or less or about 25° C. or less. The porphyrinogen compound does not need to be recovered before oxidation. The condensation may be performed with agitation or mixing. The condensation may be performed under an inert gas. The oxidizing reagents may be added to the system after completion of the oxidation.

The porphyrinogen comprises four pyrrole rings bonded through the carbonyl carbon of the aldehyde wherein the carbonyl carbon atoms of the aldehyde are bonded to the carbons adjacent to a nitrogen of the pyrrole and the structure forms a closed macrocycle with a nitrogen atom of each pyrrole located inside the macrocyclic ring. The bridging carbon atoms are unsaturated. An intermediate porphyrinogen may correspond to the formula:

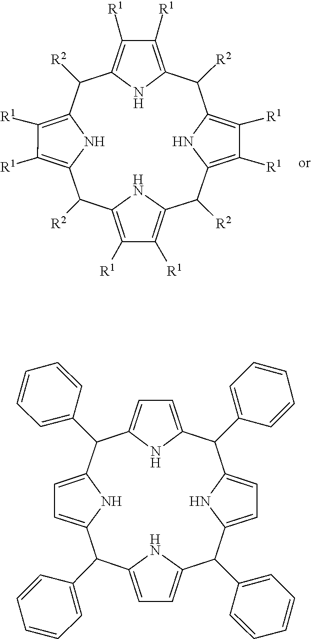

The porphyrinogen may be converted to a final porphyrin by oxidizing the porphyrinogen with an oxidizing agent to remove six hydrogens in three successive dehydrogenation steps. Such final porphyrin structures may correspond to the formula:

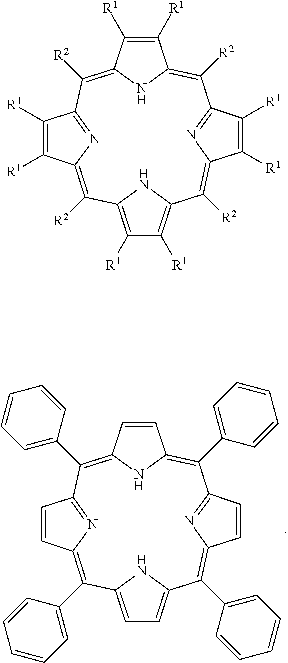

The oxidizing agent functions to remove hydrogen (e.g., 6 hydrogen) from the porphyrinogen in three successive dehydrogenation steps. The oxidizing agent may be added in a sufficient amount to convert the porphyrinogen to the porphyrin. The oxidizing agent may be one or more selected from a quinone, a peroxide, a hydroperoxide, or hydrogen peroxide. The oxidizing agent may be added in a sufficient amount to allow for three dehydrogenations. The oxidizing agent may be added a solution to stabilize the hydroperoxides. The oxidizing agent may have a concentration of about 10 weight percent solution, about 20 weight percent solution, or about 30 weight percent solution based upon a total weight of the solution containing the peroxide. The oxidizing agent may have a concentration of about 60 weight percent solution, about 50 weight percent solution, about 40 weight percent solution, or about 35 weight percent solution based on a total weight of the solution containing the peroxide. The oxidizing agent may include water or a solvent. Preferably, the oxidizing agent is anhydrous. The oxidizing agent and the porphyrinogen may be mixed a sufficient amount of time so that a porphyrin may be formed. The oxidizing agent and the porphyrinogen may be mixed for about 1 hour or more, about 3 hours or more, about 5 hours or more, or about 8 hours or more. The oxidizing agent and the porphyrinogen may be mixed for about 1 week or less, 5 days or less, 3 days or less, 1 day or less, 12 hours or less, or about 10 hours or less. The oxidizing agent may be mixed until all of the porphyrinogen is converted into porphyrin. The oxidation may be performed at any temperature at which the oxidation occurs. The oxidation may be performed at ambient temperatures. The oxidation may be performed at a temperature of greater than 0° C., at about 10° C. or greater or about 20° C. or greater. The oxidation may be performed at a temperature of about 40° C. or less or about 25° C. or less. The oxidation may be performed with agitation or mixing. The porphyrin compound may be recovered by a physical separation, for instance filtration or through the use of a centrifuge.

The porphyrin functions to be a structure that receives a metal or combines with a metal. The porphyrin may be a free base porphyrin (e.g., does not include a metal). The porphyrin may be moved to a catalyst synthesis system or be treated in a catalyst synthesis process.

The catalyst synthesis system and/or catalyst synthesis process may function to convert a free base porphyrin into a metalated porphyrin. The catalyst synthesis may convert a porphyrin into a carbonylation catalyst. The catalyst synthesis process may combine the porphyrin with one or more metalating agents. The catalyst synthesis system may convert the porphyrin in the same vessel as the porphyrin was formed (e.g., a single pot reaction). The catalyst synthesis system may convert the porphyrin in a second vessel or a third vessel. The catalyst synthesis system and/or catalyst synthesis process may react the porphyrin with one or more metalating agents to form a metalated porphyrin.

The metalating agent functions to introduce a metal into a porphyrin and preferably into a free base porphyrin to form a metalated porphyrin. The metalating agent may function to provide or donate a metal atom to a porphyrin compound. The metalating agent may include one or more metals selected from the group of aluminum, cobalt, zinc, magnesium, chromium, titanium, ruthenium, or iron. The metalating agent may be any metalating agent taught herein including those found in U.S. Pat. No. 10,428,165 in Column 53, lines 3 through 61 the teachings of which are expressly incorporated for all purposes and especially for the agents taught in '165. Preferably, the metalating agent is an aluminum, chromium, or cobalt compound. More preferably, the metalating agent is triethylaluminum. The metalating agent may provide a metal atom that may be located in a center of a macrocycle ring so that an intermediary compound is formed. (e.g., a metalated porphyrin).

The intermediary compound may be a metalated porphyrin. The metalated porphyrin functions as a precursor to a carbonylation catalyst. The metalated porphyrin may include an alkyl group or a halo group. The metalated porphyrin may include an ethyl group. The alkyl group may be removed by reacting the metalated porphyrin with a metal carbonyl.

The metal carbonyl functions to remove an alkyl substituted group from the metalated porphyrin and specifically from the metal atom within the metal porphyrin. The metal carbonyl functions to form a counteranion of a metallated porphyrin. The metal carbonyl may remove an electron from the metal porphyrin. The metal carbonyl may remove an ethyl group. The metal carbonyl may convert the metalated porphyrin to a carbonylation catalyst.

The carbonylation catalyst function to convert ethylene oxide and carbon monoxide into a lactone as is discussed herein. The lactone formed from the carbonylation reaction may be any cyclic carboxylic ester having at least one carbon atom and two oxygen atoms. For example, the lactone may be an acetolactone, a propiolactone, a butyrolactone, a valerolactone, caprolactone, or a combination thereof. Anywhere in this application where a propiolactone or lactone is used or described, another lactone may be applicable or usable in the process, step, or method. Where a propiolactone is a used or produced in the carbonylation reaction, the propiolactone may have a structure corresponding to formula 3:

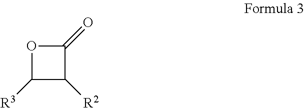

where R² and R³ are each independently selected from the group consisting of: hydrogen; C₁-C₁₅ alkyl groups; halogenated alkyl chains; phenyl groups; optionally substituted aliphatic or aromatic alkyl groups; optionally substituted phenyl; optionally substituted heteroaliphatic alkyl groups; optionally substituted 3 to 6 membered carbocycle; and optionally substituted 3 to 6 membered heterocycle groups, where R² and R³ can optionally be taken together with intervening atoms to form a 3 to 10 membered, substituted or unsubstituted ring optionally containing one or more hetero atoms; or any combination thereof.

The product stream or composition may include one or more organic compounds including a propiolactone, a polypropiolactone, succinic anhydride, polyethylene glycol, poly-3-hydroxypropionate, 3-hydroxy propionic acid, 3-hydroxy propionaldehyde, a polyester, a polyethylene, a polyether, unreacted epoxides, any derivative thereof, any other monomer or polymer derived from the reaction of an epoxide and carbon monoxide, or any combination thereof. The product stream or composition may include one or more inorganic compounds that include catalyst components such as metal carbonyls, metal carbonyl derivatives, metal centered compounds, Lewis acids, Lewis acid derivatives, or any combination thereof. A metal carbonyl derivative is a compound that includes one or more metals and one or more carbonyl groups that can be processed to form an anionic metal carbonyl component for use in a carbonylation catalyst. A Lewis acid derivative is a compound that includes one or more metal centered Lewis acids bonded to one or more undesirable compounds at the metal center that can be processed into a cationic Lewis acid for use in a carbonylation catalyst. The product stream or composition may include a catalyst that has not been spent or used up in the process of forming propiolactones. The product stream or composition may include one or more unreacted epoxides or carbon monoxide.

The product stream may include a metal centered compound coupled with one or more polymers. The one or more polymers may contain any polymerizable byproduct, reactant or both of a carbonylation reaction. The one or more polymers may contain a residue of a propiolactone, a lactone, an epoxide, an ester, an alkoxide, an oligomer, or any combination thereof. The one or more polymers may be a copolymer.

After carbonylation is conducted and the product stream or composition is formed, the one or more product streams or compositions may be subjected to one or more separation methods to remove the propiolactones, the organic compounds, the inorganic compounds, or any combination thereof. The one or more product streams or compositions may be subjected to a mechanical separation method such as nanofiltration so that larger products than the propiolactone are removed. Other separation methods may include vacuum distillation, gravity distillation, extraction, filtration, sedimentation, coagulation, centrifugation, or any combination thereof.

The one or more product streams or compositions may be subjected to a distillation process to remove any volatile compounds, such as propiolactone. The distillation process may include one or more steps of introducing a solvent that is configured to separate any propiolactone components from the composition so that the propiolactone may be distilled. The solvent may be aprotic. For example, the solvent may be a high boiling solvent that separates the propiolactone from the other components of the composition so that the propiolactone can be distilled off of the composition. The solvent may be chosen based on having a higher boiling point than the boiling point of the propiolactone. When the propiolactone is distilled off of the composition, the composition containing the organic compounds, the inorganic compounds, the metal centered compound containing the polymer, or any combination thereof may be dissolved the solvent. Whether the all or part of the composition is dissolved in the solvent, the solvent can be removed from the composition by any known separation or removal method. Distillation of the product stream may remove substantially all of the propiolactone so that the product stream or composition comprises one or more organic compounds, inorganic compounds, catalyst components, or any combination thereof that are considered byproducts to the propiolactone formation process. More details about distilling propiolactones, such as beta propiolactone, can be found in WO2010/118128A1, which is incorporated herein by reference in its entirety.

After removal of the propiolactone, the one or more product streams or compositions may be subjected to a concentration or precipitation process to separate the one or more solvents used to separate the propiolactone from the composition containing the metal centered compound.

Where a concentration step is used, the solvent used to separate the propiolactone may be distilled from the composition so that the composition is free of solvent. If the solvent is distilled from the composition, the composition may have a solid or liquid form that is free of solvent. The solvent may be further separated by one or more steps of decantation, distillation, centrifugation, or any combination thereof. The concentration step may be performed at a temperature, time, and pressure sufficient to separate the solvent from the other composition components.

Where a precipitation step is used, the method may include one or more steps of contacting the product stream or the composition and a first solvent that is insoluble in the beta propiolactone to separate the propiolactone from the product stream. For example, the first solvent may be an aprotic solvent having a boiling point that is higher than the boiling point of the propiolactone. The metal centered compound containing the polymer containing a residue of a propiolactone, an epoxide, or both may be soluble in the first solvent. In addition, a second solvent may be added that the metal centered compound containing a polymer is insoluble in so that the metal centered compound is precipitated from the first solvent and the second solvent. The second solvent may be a polar solvent. The first solvent and the second solvent may be in any ratio sufficient to precipitate the metal centered compound containing the polymer containing a residue of a propiolactone, an epoxide, or both. The first and second solvents can then be removed from the product stream by filtering off the first and second solvents from the insoluble components of the product stream by any known methods. The insoluble components may include one or more of the organic compounds, inorganic compounds, or both discussed herein and the metal centered compound. The precipitation step may be performed at ambient temperatures and pressure. In other examples, the precipitation step may be performed at a temperature of about 25 degrees C. or lower, about 20 degrees C. or lower, or about 15 degrees C. or lower. In other examples, the precipitation step may be performed at a temperature of about 0 degrees C. or more, about 5 degrees C. or more, or about 10 degrees C. or more. Additional conditions for precipitating the metal centered compound may be found in WO2015/171372A1, which is incorporated herein in its entirety.

Once the solvents used to distill the propiolactone or precipitate the metal centered compound are removed, the composition may be subject to one or more steps to remove a portion of the organic compounds, inorganic compounds, or both. In this step, an aqueous solvent can be contacted with the product stream or composition to dissolve a portion of the organic compounds, the inorganic compounds or both. The aqueous solvent may be a polar solvent. If an aqueous solvent is added, the product stream can be filtered to leave the metal centered compound and a portion of the organic compounds that are insoluble in the aqueous solvent and/or the first solvent and the second solvents described in the precipitation step.

To form a component that is useable as a carbonylation catalyst component, the metal centered compound may be decoupled from the polymer containing the residue of a propiolactone, an epoxide, or both by any means sufficient to form a Lewis acid containing a halogen or a Lewis acid containing one or more polar ligands. To decouple the metal centered compound from the polymer containing the residue of a propiolactone, an epoxide, or both, the composition or product stream may be subjected to a thermolysis step or a contacting step with an acid compound. The decoupling steps functions to remove the polymer from the metal centered compound and to bond or connect a halogen or a polar ligand to the metal centered compound so that the Lewis acid containing a halogen or a polar ligand are formed. Forming the Lewis acid containing a halogen or a polar ligand are precursors to forming the recycled components for the regenerated catalyst.

In one step of decoupling the polymer containing the residue of a propiolactone, an epoxide, or both and the metal centered compound, a thermolysis step or process may be performed to make one or more unsaturated acids. The thermolysis step functions to denature the polymer, any organic compounds, or both in the product stream to form an unsaturated acid. For example, an unsaturated acid may be one or more of acrylic acid, polyacrylic acid, or any combination thereof. Subsequent to subjecting the composition to thermolysis, the composition may be contacted with an acid compound and a solvent to form the Lewis acid containing a halogen. When the acid compound is contacted with the composition, any organic compounds contained within the metal centered compound may be decoupled from the metal centered compound, and the acid compound may deliver a halogen to the metal centered compound to form a Lewis acid containing a halogen. The thermolysis step may be performed at pressure of about 1 torr or more, about 25 torr or more, or about 50 torr or more. The thermolysis step may be performed at a pressure of about 1000 torr or less, about 500 torr or less, or about 100 torr or less. The thermolysis step may be performed at a temperature of about 130 degrees C. or more, about 160 degrees C. or more, or about 190 degrees C. or more. The thermolysis step may be performed at a temperature of about 300 degrees C. or less, about 250 degrees C. or less, or about 220 degrees C. or less. The thermolysis step may be performed for about 15 seconds or more, about 15 minutes or more, or about 30 minutes or more. The thermolysis step may be performed for about 24 hours or less, about 12 hours or less, or about 1 hour or less. Additional conditions or steps for the thermolysis process can be found in U.S. Pat. No. 10,065,91461, which is incorporated herein by reference in its entirety. The thermolysis step may be paired with other separation techniques described herein so that the one or more of the organic compounds, the inorganic compounds, the solvents, or both are removed from the composition that includes the metal centered compound, Lewis acid containing the halogen or polar ligand, or both. Before, after, or during the forming of the Lewis acid containing a halogen, the composition may be subjected to any filtering or removing step sufficient to remove any remaining organic compounds, inorganic compounds, or both that may interfere with the formation of the regenerated catalyst.

In another step of decoupling the polymer containing the residue of a propiolactone, an epoxide, or both and the metal centered compound, the feed stream or composition may be contacted with an acid compound to decouple the polymer containing the residue of the propiolactone, an epoxide, or both and the metal centered compound. The contacting of the acid compound with the feed stream or composition functions to cleave or decouple the polymer containing the residue of a propiolactone, an epoxide, or both and the metal centered compound. In one example, the composition, an acid compound, and a solvent may be contacted so that the solvent dissolves the metal centered compound and the acid compound cleaves or decouples the polymer containing a residue of the propiolactone, an epoxide, or both from the metal centered compound. In this contacting step to cleave or decouple the polymer and the metal centered compound, the components may be stirred by any agitation means known to a skilled artisan, such as utilizing a magnetic stir bar. The components may be stirred for about 1 minute or more, about 30 minutes or more, or about 45 minutes or more. The components may be stirred for about 180 minutes or less, about 120 minutes or less, or about 60 minutes or less. The contacting step may be performed at ambient conditions including at least ambient temperature and pressure. Some components, such as a portion of the organic compounds, the inorganic compounds, or both, may not be soluble in the solvent, and the composition may be subjected to a step of filtering the insoluble components so that the composition includes the Lewis acid containing the halogen, the solvent, and any unreacted acid compounds remaining. This filtering step may include one or more of gravity filtration, or any combination thereof. In an additional step, the composition may be subjected to a step of removing the acid compound, the solvent, or both so that a solid precipitate of the composition comprising the Lewis acid containing the halogen remains. The removing step may include one or more vacuum filtration, distillation, or any combination thereof. After the removal step or the filtration step, the composition including a Lewis acid containing a halogen may be subjected to one or more steps to regenerate the catalyst.

In another example, a contacting step is performed that uses an initial solvent to decouple the polymer and the metal centered compound and uses another subsequent solvent to precipitate a Lewis acid containing a halogen. In this example, the composition, an initial solvent, and an acid compound may be contacted so that the initial solvent dissolves the metal centered compound and the acid compound cleaves or decouples the polymer containing a residue of the propiolactone, an epoxide, or both. In this contacting step to cleave or decouple the polymer and the metal centered compound, the components may be stirred by any agitation means known to a skilled artisan. The components may be stirred for about 1 minute or more, about 30 minutes or more, or about 45 minutes or more. The components may be stirred for about 180 minutes or less, about 120 minutes or less, or about 60 minutes or less. The contacting step may be performed at ambient conditions including at least ambient temperature and pressure. Some components, such as a portion of the organic compounds, the inorganic compounds, or both, may not be soluble in the initial solvent, and the composition may be subjected to a step of filtering the insoluble components so that the composition includes the Lewis acid containing the halogen, the initial solvent, and any unreacted acid compounds remaining. This filtering step may include one or more of gravity filtration, centrifugation, decantation, or any combination thereof. After filtering, a subsequent solvent that is miscible in the initial solvent may be contacted with the composition including the Lewis acid containing the halogen, the initial solvent, and any unreacted acid compounds so that the Lewis acid containing the halogen is precipitated from the other components of the composition. The initial solvent and the subsequent solvent may be described as first and second solvents. The initial solvent may be a polar solvent, and the subsequent solvent may be an aprotic solvent. The initial solvent and the polar solvent may be miscible in each other. The initial solvent and the subsequent solvent may be added to the composition in a ratio sufficient to precipitate the Lewis acid containing the halogen, one or more organic compounds, or both. The precipitated composition including the Lewis acid containing the halogen may be subjected to a filtering step to remove the initial solvent, the subsequent solvent, any remaining organic compounds, and any unreacted acid compound. The filtering step may include one or more of gravity filtration, centrifugation, decantation, or any combination thereof. After the removal step or the filtration step, the composition including a Lewis acid containing a halogen may be subjected to one or more steps to regenerate the catalyst.

Another method may be to utilize an extraction technique by contacting an acid compound with a combination of solvents to form a multi-phase composition that can distinctly separate undesirable components from the Lewis acid containing a halogen. To initiate, the composition may be contacted with an acid compound, aqueous solvent, and an organic solvent to form a slurry with multiple phase layers including an aqueous liquid phase, an organic liquid phase, and a precipitate. Upon contacting the acid compound with the metal centered compound containing the polymer containing a residue of the propiolactone, an epoxide, or both, the acid compound decouples the polymer and the metal centered compound and bonds the halogen to the metal centered compound to form the Lewis acid containing the halogen. The aqueous solvent may be configured to dissolve one or more of the organic compounds, the polymer containing the residue of the propiolactone, an epoxide, or both, the inorganic compounds, or any combination thereof. The organic solvent may be configured to dissolve the Lewis acid containing a halogen. The precipitate may include any compound that is insoluble in the aqueous solvent or the organic solvent. In some examples, no precipitate is formed. In this contacting step to decouple the polymer and the metal centered compound, the components may be stirred by any agitation means known to a skilled artisan. The components may be stirred for about 1 minute or more, about 30 minutes or more, or about 45 minutes or more. The components may be stirred for about 180 minutes or less, about 120 minutes or less, or about 60 minutes or less. The contacting step may be performed at ambient conditions including at least ambient temperature and pressure. The components may be contacted for any period of time sufficient to form an organic phase, an aqueous phase, or any combination thereof. Once the phases are formed, the aqueous phase is removed so that the organic phase remains. The aqueous phase may be removed by simple extraction or any other known technique sufficient to remove the aqueous phase from the precipitate, the organic phase, or both. If a precipitate is present, the precipitate may be filtered from the organic phase so that the composition includes the organic solvent and the Lewis acid containing the halogen. Subsequently, the organic solvent may be removed from the Lewis acid containing the halogen by any suitable filtration or removal step, such as vacuum filtration. After separating the Lewis acid from other components of the composition, the Lewis acid containing the halogen may be subjected to steps to regenerate the carbonylation catalyst without interference from other compounds.

When the acid compound is an exchange resin, an acid ion exchange resin or an anion exchange resin may be used to form the Lewis acid containing halogen. The acid ion exchange resin functions to decouple the metal centered compound and the polymer and to physically separate the metal centered compound from the composition. The anion exchange resin functions to exchange the polymer of the metal centered compound with a halogen to form the Lewis acid containing a halogen.

Where the acid ion exchange resin is used, the acid ion exchange resin may be used to bond with the metal centered compound and, subsequently, another acid compound may be used to form the Lewis acid containing the halogen. The metal centered compound, a polar solvent, and an acid ion exchange resin may be contacted so that the metal centered compound and the polymer containing the propiolactone are cleaved or decoupled, and the acid ion exchange resin may bond with the metal centered compound. The acid ion exchange resin may be in the form of a bead, gel, or solid support. The polar solvent may be configured to dissolve any components that have aqueous solubility, such as any of the inorganic compounds, the organic compounds, or both. The polar solvent may be an aqueous solvent, such as water. The combination of the metal centered compound and the exchange resin may be insoluble in the solvent. Through a filtration or a removing step, the components dissolved in the aqueous solvent and the aqueous solvent may be removed from the composition so that a precipitate of the compounds that do not have aqueous solubility remain, for example, the acid ion exchange resin tethered to the metal centered compound, any organic compounds not having aqueous solubility, or both. Subsequently, the acid ion exchange resin tethered to the metal centered compound may be separated from any remaining organic compounds so the composition includes acid ion exchange resin tethered to the metal centered compound. One method could include dissolving the organic compounds in a solvent suitable for dissolving the organic compounds, like an aprotic solvent, and filtering the solvent and organic compounds from the composition through gravity filtration. After separating other components from the acid ion exchange resin tethered to the metal centered compound, another acid compound could be contacted with the composition to decouple the metal centered compound from the exchange resin and deliver a halogen to the metal centered compound so that a Lewis acid containing a halogen is formed. Once the Lewis acid containing the halogen is formed, the acid ion exchange resin may be recovered and used for additional decoupling steps of the metal centered compound and the polymer containing the residue of the propiolactone, an epoxide, or both.

In other examples using the anion exchange resin, the anion exchange resin may directly decouple the metal centered compound and the polymer containing the residue of a propiolactone, an epoxide, or both, and the Lewis acid containing the halogen may be formed without adding an additional acid compound. In this case, a polar solvent may dissolve the metal centered compound and other components so that the composition can be simply moved across an anion exchange resin that is a solid support. In other examples, the anion exchange resin may be a bead. In other words, the metal centered compound may be dissolved in the solvent, and then moved over the anion exchange resin so that the anion exchange resin bonds with the polymer containing the residue of the propiolactone, an epoxide, or both, one or more organic compounds, or both. The anion exchange resin may separate the polymer, the one or more organic compounds, or both from the composition so that the composition includes the polar solvent and the Lewis acid containing the halogen. After contacting with metal centered compound with the exchange resin to form the Lewis acid containing a halogen, the anion exchange resin containing the one or more organic compounds, the polymer, or both may be removed from the composition. In other examples, the composition that is liquid and including the metal centered compound containing a polymer may be inserted into a vessel containing the anion exchange resin, run across the anion exchange resin in the vessel to exchange the polymer and the halogen to form the Lewis acid containing a halogen, and exit the vessel at an outlet so that the liquid composition includes a Lewis acid containing the halogen. The other components that are not captured on the anion exchange resin may be removed by gravity filtration, vacuum filtration, or any combination thereof. After separating the anion exchange resin and the composition, the solvent may be separated from the composition including the Lewis acid containing the halogen by filtration, such as vacuum filtration, or any combination thereof. After forming the Lewis acid containing a halogen from one of the anion exchange resins, the Lewis acid containing the halogen may be subjected to one or more steps to regenerate the carbonylation catalyst.

In either case of using an acid ion exchange resin or an anion exchange resin, the contacting conditions may be as follows to sufficiently form either an acid ion exchange resin tethered to the metal centered compound, the Lewis acid containing a halogen, or both: The components may be stirred by any agitation means known to a skilled artisan. The components may be stirred for about 1 minute or more, about 45 minutes or more, or about 2 hours or more. The components may be stirred for about 24 hours or less, about 12 hours or less, or about 4 hours or less. The contacting step may be performed at ambient conditions including ambient temperature and pressure. Further, when the acid ion exchange tethered to the metal centered compound is contacted with another acid compound, the contacting conditions may be similar in regards to agitation, time, temperature, pressure, or any combination thereof.

The filtering or removing steps taught herein function to remove from the composition any unwanted components that may interfere with the formation of a reclaimed or regenerated catalyst. For example, one or more of solvents, polymers, exchange resins, unreacted acid compounds, inorganic compounds, organic compounds, or any combination thereof may be removed from the composition so that the carbonylation catalyst may be regenerated from the Lewis acid containing a halogen or a polar ligand and have catalytic activity with one or more of succinic anhydride, propiolactone, or an epoxide. The filtering or removing steps may include one or more of vacuum filtration, gravity filtration, centrifugation, decantation, precipitation, phase layer extraction, or any combination thereof. The filtering or removing steps may utilize any method sufficient to separate one or more of solvents, polymers, exchange resins, unreacted acid compounds, inorganic compounds, organic compounds, or any combination thereof and the Lewis acid containing the halogen or a polar ligand, the metal centered compound, or any combination thereof. The filtering or removing steps may remove a single type of compound at a time, such as a precipitate, or may remove a collection of compounds at a time, such as all components dissolved in a solvent. The filtering or removing steps may include forming multiple phases including one or more of one or more organic phases, an aqueous phase, a solid phase (i.e., a precipitate), one or more gaseous or vapor phases, or any combination thereof. The one or more separation or removal steps/methods described herein may be performed at any temperature, pressure, agitation rate, time, or any combination thereof sufficient to separate or remove any undesirable component from the composition including the metal centered compound, the Lewis acid containing the halogen or polar ligand, or any combination thereof.

Either after or during the decoupling steps described herein, the method may include one or more regeneration steps that function to decouple or cleave either a polymer containing a residue of a propiolactone, an epoxide, or both or a halogen from the metal centered compound and/or Lewis acid. The one or more regeneration steps function to modify a metal centered compound and/or a Lewis acid to a regenerated carbonylation catalyst comprising a Lewis acid and a metal carbonyl.

The regeneration step may include contacting the metal centered compound containing a polymer containing a residue of a propiolactone, an epoxide, or both or the Lewis acid containing a halogen with a polar ligand, a metal carbonyl additive, or both. The metal carbonyl additive may contain at least a metal carbonyl that is anionic and a cationic group that is configured to cleave and bond with the polymer of the metal centered compound. The cationic group may be one or more of an alkali metal, Ph₃Si—, R₃Si—, any counterion sufficient to ionically bond and/or balance the metal carbonyl, or any combination thereof, where R is independently selected from a phenyl, halophenyl, hydrogen, alkyl, alkylhalo, alkoxy, or any combination thereof. In examples where the metal carbonyl additive cleaves or decouples the polymer, the polymer may couple with the cationic group, and the polymer and cationic group could be removed via any filtration or removal means described herein. In examples where the metal carbonyl cleaves the halogen from the metal centered compound and is contacted with the polar compound, the halogen bonds with the cationic group of the metal carbonyl additive and the Lewis acid containing the polar compound is formed. Additional polymers can be removed by any other removal or separation steps described herein. After the metal carbonyl additive cleaves or decouples the polymer, the Lewis acid may combine with the polar ligand to form a cationic species. The Lewis acid containing the polar ligand then contacts with the metal carbonyl that is anionic of the metal carbonyl additive and forms the regenerated carbonylation catalyst. An example of contacting a polar ligand, a metal carbonyl additive, and the Lewis acid containing a halogen is shown in reaction (I).

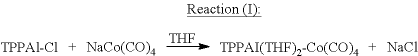

Where TPPAI-CI (tetraphenyl porphyrin aluminum chloride complex) is the Lewis acid containing a halogen; NaCo(CO)₄ is the metal additive; THF (tetrahydrofuran) is a polar ligand and/or a polar solvent; and TPPAI(THF)₂-Co(CO)₄ is the regenerated carbonylation catalyst. The steps to regenerate the catalyst may be performed under conditions that are moisture and oxygen free. For example, the regeneration steps may be performed within a dry glove box, on a Schlenk line, or in a reactor under an inert atmosphere. The regeneration steps may be performed under a nitrogen, argon, or any other inert gas. During the regeneration steps, the Lewis acid, the polar ligand, the metal carbonyl, or any combination thereof may be contacted and agitated by stirring for a period of time sufficient to form the regenerated catalyst. The period of time for stirring the components may be about 5 minutes or more, about 30 minutes or more, about 60 minutes or more. The period of time for stirring the components may be about 24 hours or less, about 12 hours or less, or about 6 hours or less. The components in the regeneration step may be completed under ambient temperature and/or pressure. Additional steps to make the regenerated catalyst can be found in U.S. Pat. No. 6,852,865B2 and U.S. Pat. No. 8,481,75661, both of which are included herein by reference in their entirety.

The acid compound may function to cleave the polymer from the metal centered compound and to deliver a halogen to the metal centered compound. The acid compound may contain a halogen such as fluorine, chlorine, bromine, iodine, or a combination thereof. The acid compound may be any compound capable of delivering a halogen to the metal centered compound to form a Lewis acid containing a halogen. The acid compound may be HF, HCl, HBr, Hl, or any combination thereof. When mixed or dissolved with an aqueous solvent, such as water, the acid compound may have a molarity of 1 or more, 1.5 or more, 1.8 or more, or 2.0 or more. The acid compound may have a molarity of 3.5 or less, 3.0 or less, 2.8 or less, or 2.5 or less. The acid compound may be an exchange resin, such as an acid ion exchange resin, an anion exchange resin, or both. The acid ion exchange resin, the anion exchange resin, or both may include one or more of a quaternary ammonium compound, a sulfonic group, or any combination thereof. The acid ion exchange resin, the anion exchange resin, or both may be polystyrene based, macro-porous, styrene-divinyl benzene copolymer, or any combination thereof. Any of the exchange resins may be anionic or cationic. The acid compound may be any compound sufficient to cleave or decouple a polymer from a metal centered compound and deliver a halogen compound to the metal centered compound to form a Lewis acid containing a halogen. The acid compound may be a metal carbonyl additive that comprises one or more cationic trisubstituted silyl groups having a structure corresponding to R₃Si, and one or more anionic metal groups, where R is independently selected from a phenyl, halophenyl, hydrogen, alkyl, alkylhalo, alkoxy, or any combination thereof.

The carbonylation catalyst as described herein functions to catalyze a reaction of an epoxide and carbon monoxide to produce one or more propiolactones and other products. The carbonylation catalyst includes at least a metal carbonyl that is anionic and a Lewis acid that is cationic.

The metal carbonyl of the carbonylation catalyst functions to provide the anionic component of the carbonylation catalyst. The carbonylation catalyst may include one or more, two more, or a mixture of metal carbonyls. The metal carbonyl may be capable of ring-opening an epoxide and facilitating the insertion of CO into the resulting metal carbon bond. In some examples, the metal carbonyl may include an anionic metal carbonyl moiety. In other examples, the metal carbonyl compound may include a neutral metal carbonyl compound. The metal carbonyl may include a metal carbonyl hydride or a hydrido metal carbonyl compound. The metal carbonyl may be a pre-catalyst which reacts in situ with one or more reaction components to provide an active species different from the compound initially provided. The metal carbonyl includes an anionic metal carbonyl species. in some examples, the metal carbonyl may have the general formula [Q_{d}M′ₑ(CO)_{w}]^{y+}, where Q is an optional ligand, M′ is a metal atom, d is an integer between 0 and 8 inclusive, e is an integer between 1 and 6 inclusive, w is a number such as to provide the stable anionic metal carbonyl complex, and y is the charge of the anionic metal carbonyl species. The metal carbonyl may include monoanionic carbonyl complexes of metals from groups 5, 7 or 9 of the periodic table or dianionic carbonyl complexes of metals from groups 4 or 8 of the periodic table. The metal carbonyl may contain cobalt, manganese, ruthenium, or rhodium. Exemplary metal carbonyls may include [Co(CO)₄]⁻, [Ti(CO)e]²⁻, [V(CO)₆]⁻, [Rh(CO)_{4]}⁻, [Fe(CO)₄]²⁻, [Ru(CO)₄]²⁻, [Os(CO)_{4]}²⁻, [Cr₂(CO)₁₀]²⁻, [Fe₂(CO)₈]²⁻, [Tc(CO)₅]⁻, [Re(CO)₅]⁻, and [Mn(CO)₅]⁻. The metal carbonyl may be a mixture of two or more anionic metal carbonyl complexes in the carbonylation catalysts used in the methods.

A metal carbonyl additive functions to deliver a metal carbonyl to a Lewis acid that is suitable to combine and form the regenerated carbonylation catalyst. The metal carbonyl additive may function to decouple a halogen or a polymer containing a residue of a propiolactone, an epoxide, or both from a metal centered compound to form the regenerated carbonylation catalyst that includes the Lewis acid and metal carbonyl combination. The metal carbonyl additive includes at least a metal carbonyl as described herein and a cationic compound. The cationic compound may include lithium, sodium, potassium, rubidium, cesium, beryllium, magnesium, calcium, strontium, barium, radium, or any combination thereof. The metal carbonyl additive may be a salt. The metal carbonyl additive may be a silicon salt in the form of R₃Si—, where R is independently selected from a phenyl, halophenyl, hydrogen, alkyl, alkylhalo, alkoxy, or any combination thereof.

The Lewis acid functions to provide the cationic component of the carbonylation catalyst. The Lewis acid may be a metal centered compound, a metal complex, or both that is configured to be anionically balanced by one or more metal carbonyls. The Lewis acid component of the carbonylation catalyst may include a dianionic tetradentate ligand. The Lewis acid may include one or more porphyrin derivatives, salen derivatives, dibenzotetramethyltetraaza[14]annulene (tmtaa) derivatives, phthalocyaninate derivatives, derivatives of the Trost ligand, tetraphenylporphyrin derivatives, tetramethyl-tetra-aza-annulene type, and corrole derivatives. In some examples, where the carbonylation catalysts used in the disclosed methods include a cationic Lewis acid including a metal complex, the metal complex has the formula [(L^{c})ᵥM_{b}]^{Z+}, where:
L is a ligand where, when two or more L are present, each may be the same or different;
M is a metal atom where, when two M are present, each may be the same or different;
v is an integer from 1 to 4 inclusive;
b is an integer from 1 to 2 inclusive; and
z is an integer greater than 0 that represents the cationic charge on the metal complex.

In other examples, the Lewis acid or metal centered compound may have a structure of metal complex I or II. Where the Lewis acid has the metal complex I, the metal complex may be the following configuration:

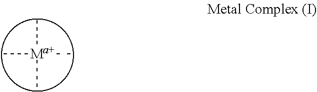

where

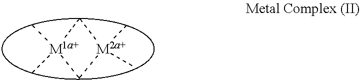

is a multidentate ligand;
M is a metal atom coordinated to the multidentate ligand; and
a is the charge of the metal atom and ranges from 0 to 2. and In certain embodiments, provided metal complexes conform to metal complex II.

In other examples, the Lewis acid may have the metal complex having the formula of metal complex II:

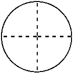

Where a is as defined above (each a may be the same or different),
M1 is a first metal atom;
M2 is a second metal atom; and

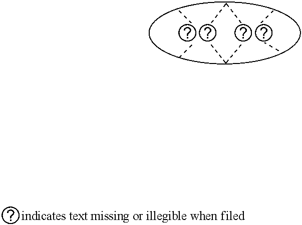

comprises a multidentate ligand system capable of coordinating both metal atoms.

In the decoupling, separation, removal, and regeneration steps of the present disclosure, the solvent may function to dissolve or precipitate one or more of the components discussed herein. The solvent may be selected from any solvent discussed herein or a mixtures of solvents. The solvent may be polar, nonpolar, aprotic, protic, aqueous, organic, or any combination thereof.

The solvent may be an aprotic solvent that functions to dissolve one or more compounds that lack one or more protic elements. For example, the aprotic solvent may be configured to dissolve the one or more organic compounds, the metal compound containing a polymer, or both. The aprotic solvent may separate one or more lactones from a product stream of a carbonylation reaction. The aprotic solvent may be soluble in one or more other nonpolar or polar solvents. The aprotic solvent may be insoluble with one or more of a carboxylic acid, a lactone, a or any combination thereof. The aprotic solvent may be a high boiling solvent to facilitate filtering or removing of volatile components (e.g., lactones) of the composition. The aprotic solvent may be combined with a second solvent that is miscible in the aprotic solvent to precipitate components that are insoluble in the second solvent. The aprotic solvent may be selected to form an organic phase layer that is distinct from an aqueous phase layer, a precipitate, or both. The aprotic solvent may be one or more of hexane, heptane, nonane, decane, tetrahydrofuran, methyltetrahydrofuran, diethyl ether, sulfolane, toluene, pyridine, diethyl ether, 1,4-dioxane, acetonitrile, ethyl acetate, dimethoxy ethane, acetone, chloroform, dichloromethane, or any other hydrocarbyl capable of separating a lactone from a composition, or any combination thereof.

The solvent may be a polar solvent that functions to dissolve one or more components of the composition that have polar features. The polar solvent may function to dissolve a Lewis acid and to coordinate a metal center of a Lewis acid. The polar solvent may be miscible in one or more other aprotic or protic solvents. The polar solvent may be configured to be miscible in one or more second solvents and be insoluble in a component dissolved in the second solvent so the component dissolved in the second solvent is precipitated. The polar solvent may dissolve one or more of the inorganic components, the organic compounds, the metal centered compound containing a polymer, the Lewis acid, the metal carbonyl, the metal carbonyl additive, or any combination thereof. The polar solvent may be selected to form an aqueous phase layer or an organic phase layer that is distinct from another aqueous phase layer, another organic phase layer, a precipitate, or some combination of different phase layers. The polar solvent may be one or more of water, methanol, ethanol, propanol, tetrahydrofuran, methyltetrahydrofuran, diethyl ether, sulfolane, pyridine, diethyl ether, 1,4-dioxane, acetonitrile, ethyl acetate, dimethoxy ethane, acetone, dichloromethane, or any combination thereof.

Several techniques have been theorized to illustrate the teaching of the present disclosure. Each teaching is simply an example of the disclosure and is not intended to limit the teachings to any single technique.

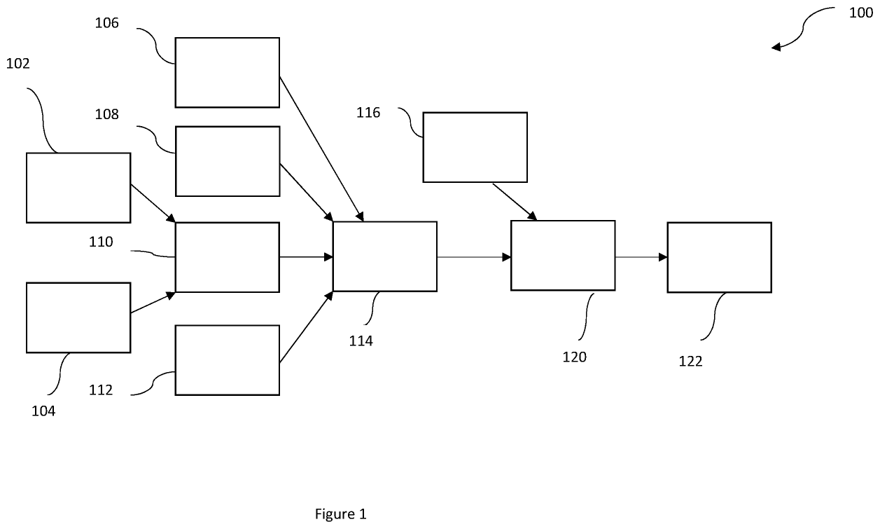

FIG. 1 illustrates a porphyrin system and process 100 for producing a porphyrin 122. The process 100 begins by mixing a solvent 112, a salt 108, a pyrrole or substituted pyrrole 102 and an aliphatic or aromatic aldehyde 104 together into a vessel 110. A catalyst 106 is then added into the vessel 110 forming a porphyrinogen 114. The porphyrinogen 114 is reacted with an oxidizing agent 116 in a step 120 to form a porphyrin 122.

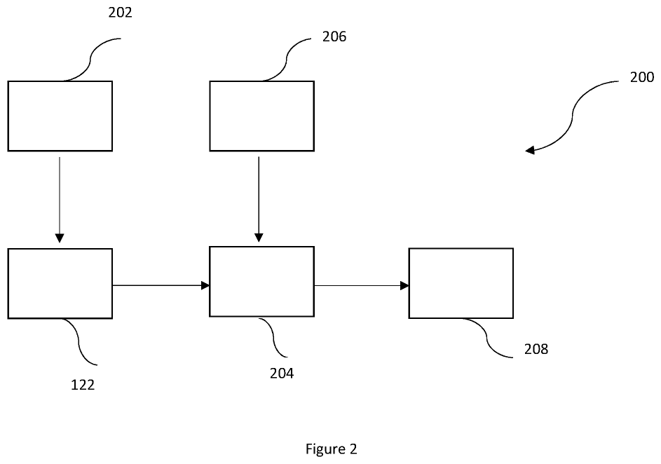

FIG. 2 illustrates a catalyst synthesis system and process 200 forming a carbonylation catalyst 208 from a porphyrin 122. The porphyrin 122 is combined with a metalating agent 202 forming an intermediary component 204 that is reacted with a metal carbonyl 206 to form carbonylation catalyst 208.

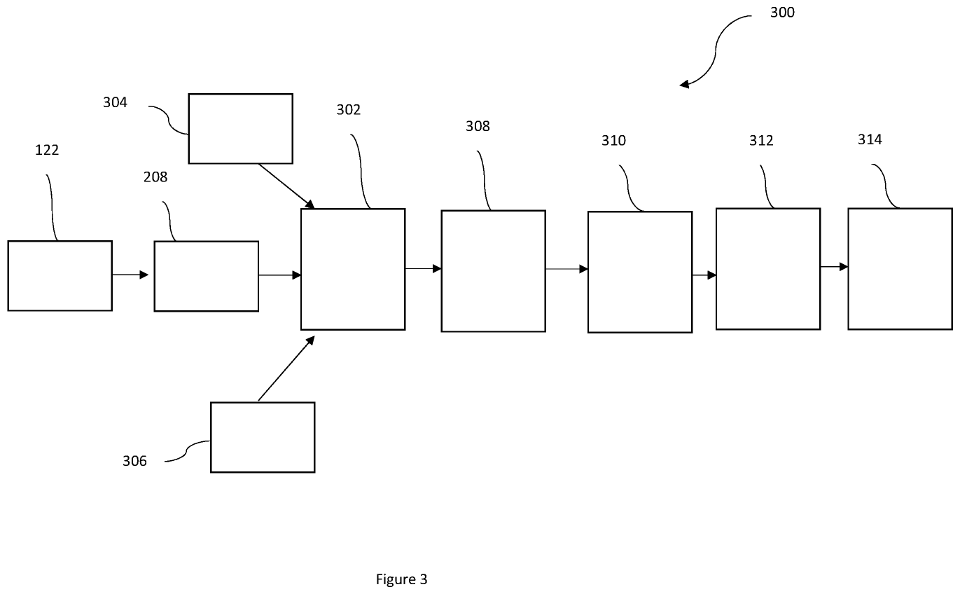

FIG. 3 illustrates a polyacrylic acid and/or a superabsorbent polymer system and method 300 to create a polyacrylic acid and/ or a superabsorbent polymer. The system and method 300 begin by forming a porphyrin 122 and converting the porphyrin into a carbonylation catalyst 208. The carbonylation catalyst 208 is mixed in a central reactor 302 with ethylene oxide 304 and carbon monoxide 306. Beta propiolactone 308 flows from the central reactor 302. The beta propiolactone 308 undergoes pyrolysis 310 and is converted into glacial acrylic acid 312. Alternatively, beta propiolactone 308 is converted to a superabsorbent polymer 314 comprising one or more polymer chains having ring opened beta propiolactone and/or substituted beta propiolactone units and having on one end of the chains a residue of an anion covalently bonded to the one end of the polymer chains.

### ENUMERATED EMBODIMENTS

Variation 1 may comprise a method comprising: (a) combining a solvent, salt, pyrrole or substituted pyrrole and an aliphatic or aromatic aldehyde in a vessel; (b) charging the vessel with a catalyst to form an porphyrinogen of the pyrrole or substituted pyrrole; and (c) oxidizing the formed porphyrinogen of the pyrrole or substituted pyrrole with an oxidizing agent to form a porphyrin; wherein the porphyrin comprises a residue of the pyrrole or substituted pyrrole having the aliphatic or aromatic groups derived from the aliphatic or aromatic aldehyde pendant from the heterocycle.

Variation 2 may comprise the method of variation 1, and wherein the catalyst is one or more selected from the group consisting of BF₃-etherate, TFA, p-CH₃C₆H₄SO₃H·H₂O, CH₃SO₃H, SbF₅, GeBr₄, PBr₅, BBr₃, TiBr₄, CCl₃CO₂H, CuCl₂, AlCl₃, MgBr₂, TiCl₄, GaCl₂, SnCl₄, FeCl₃, HCl, SiCl₄, BCl₃, TeCl₄, C₆F₅CO₂H, Gel₄, BEt₃.

Variation 3 may comprise the method of any of variations 1-2, and wherein the catalyst is one or more selected from the group consisting of BF₃-etherate, TFA, p-CH₃C₆H₄SO₃H·H₂O, CH₃SO₃H, SbF₅, GeBr₄, PBr₅, BBr₃, TiBr₄, CCl₃CO₂H, CuCl₂, AlCl₃, MgBr₂, TiCl₄, GaCl₂, SnCl₄, FeCl₃, HCl, SiCl₄, BCl₃, or TeCl₄.

Variation 4 may comprise the method of any of variations 1-3, and wherein the catalyst is one or more selected from the group consisting of BF3-etherate, TFA, or p-CH3C6H4SO3H·H2O.

Variation 5 may comprise the method of any of variations 1-4, and wherein the catalyst is boron trifluoride etherate. Variation 6 may comprise the method of any of variations 1-5, and wherein the salt is one or more of the salts selected from the group consisting of LiCl, NaCl, KCl, CsCl, MgCl₂, CaCl₂, NH₄Cl, Me₄NCl, NaBPh₄, BaBr, Kl, Na₂SO₄, MgSO₄, CaSO₄, NaBF₄, Me₄NBF₄, or KF.

Variation 7 may comprise the method of any of variations 1-6, and wherein the salt is one or more of the salts selected from the group consisting of LiCl, NaCl, KCl, CsCl, MgCl₂, CaCl₂, Paraquat 2Cl—, NH₄Cl, Me₄NCl, NaBPh₄, BaBr, or Kl.

Variation 8 may comprise the method of any of variations 1-7, and wherein the salt is one or more of the salts selected from the group consisting of NaCl, Me4NCl, or NaBPh4.

Variation 9 may comprise the method of any of variations 1-8, and wherein the salt is a metal salt.

Variation 10 may comprise the method of variation 9 or any of variations 1-9, and wherein the metal salt includes an anion selected from the group consisting of Cl—, Ph4B—, Br—, or I—.

Variation 11 may comprise the method of any of variations 1-10, and wherein the salt is sodium chloride.

Variation 12 may comprise the method of any of variations 1-11, and wherein the solvent is a halogenated solvent.

Variation 13 may comprise the method of any of variations 1-12, and wherein the solvent has a boiling point above ambient temperature.

Variation 14 may comprise the method of any of variations 1-13, and wherein the solvent is dichloromethane.

Variation 15 may comprise the method of any of variations 1-14, and wherein the oxidizing agent is a peroxide.

Variation 16 may comprise the method of variation 15 or any of variations 1-15, and wherein the peroxide is a hydroperoxide.

Variation 17 may comprise the method of variation 15 or any of variations 1-16, and wherein the oxidizing agent is hydrogen peroxide or tert-butyl hydroperoxide.

Variation 18 may comprise the method of any of variations 1-17, and wherein the pyrrole or substituted pyrrole, the aliphatic or aromatic aldehyde, the salt, and solvent are mixed before the catalyst is added.

Variation 19 may comprise the method of variation 18 or any of variations 1-18, and wherein the porphyrinogen is mixed with the catalyst.

Variation 20 may comprise the method of variation 19 or any of variations 1-19, and wherein the porphyrinogen is mixed for about 5 minutes or more and about 15 minutes or less.

Variation 21 may comprise the method of any of variations 1-20, and wherein the porphyrinogen is mixed with the oxidizing agent for 8 hours or more and 24 hours or less.

Variation 22 may comprise the method of any of variations 1-21, and wherein the method is performed at room temperature.

Variation 23 may comprise the method of any of variations 1-22, and further comprising extracting the oxidizing agent with a polar aprotic solvent before being mixed with the porphyrinogen.

Variation 24 may comprise the method of any of variations 1-23, and further comprising extracting an aqueous hydrogen peroxide solution with a polar aprotic solvent to remove water and form an anhydrous peroxide solution.

Variation 25 may comprise the method of variation 24 or any of variations 1-24, and wherein the solvent is an either or preferably diethyl ether.

Variation 26 may comprise the method of any of variations 1-25, and the porphyrin comprises tetraphenylporphyrin.

Variation 27 may comprise the method of any of variations 1-26, and further comprising a step of contacting the porphyrin with a metalating agent.

Variation 28 may comprise the method of variation 27 or any of variations 1-27, and wherein the metalating agent is an aluminum or chromium compound.

### EXAMPLES

The following examples are provided to illustrate the invention, but are not intended to limit the scope thereof. All parts and percentages are by weight unless otherwise indicated.

Example 1: A 250 mL flask is oven dried. The flask is charged with 58 mg of NaCl (1 mmol, 0.4 equiv), 100 mL dichloromethane, 0.7 mL of freshly distilled pyrrole (10 mmol, 4 equiv), and 1.0 mL benzaldehyde (10 mmol, 4 equiv) forming a mixture. The mixture is stirred for 5 minutes forming a reaction mixture. After the mixture is stirred BF 3 etherate (1 mmol, 0.4 equiv) is added to the reaction mixture and the reaction mixture is stirred for 10 minutes. The stirred reaction mixture is charged with 10 mL of a hydrogen peroxide solution (30% wt, 40 equiv, 0.10 mol) in one dose and then stirred for 18 hours. The reaction mixture is filtered through a silica plug and then concentrated in vacuo. The solid is triturated with 20 mL of MeOH, filtered, and washed with MeOH to obtain a crystalline purple solid.

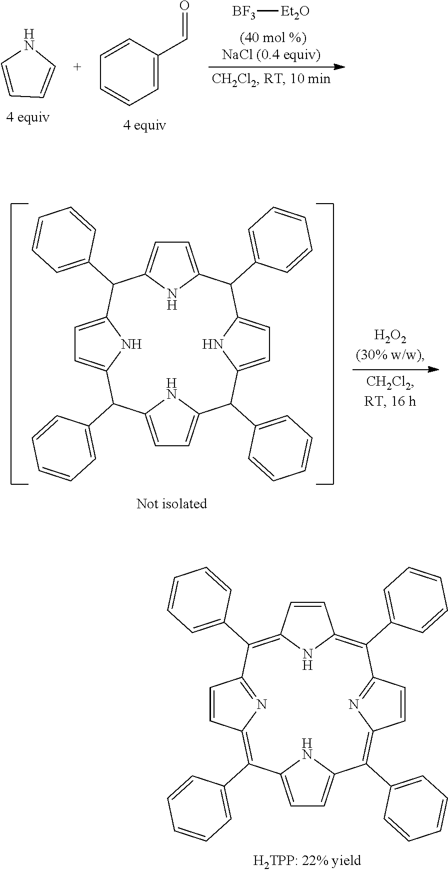

1. A method comprising:
a. combining a solvent, salt, pyrrole or substituted pyrrole, and an aliphatic or aromatic aldehyde in a vessel;
b. charging the vessel with a catalyst to form a porphyrinogen of the pyrrole or substituted pyrrole; and
c. oxidizing the formed porphyrinogen of the pyrrole or substituted pyrrole with an oxidizing agent to form a porphyrin, wherein the oxidizing agent is peroxide;
wherein the porphyrin comprises a residue of the pyrrole or substituted pyrrole having the aliphatic or aromatic groups derived from the aliphatic or aromatic aldehyde pendant from the heterocycle; and
wherein the porphyrin is free of hydroquinone byproducts.

2. The method of claim 1, wherein the catalyst is one or more selected from the group consisting of BF₃-etherate, TFA, p-CH₃C₆H₄SO₃H·H₂O, CH₃SO₃H, SbF₅, GeBr₄, PBr₅, BBr₃, TiBr₄, CCl₃CO₂H, CuCl₂, AlCl₃, MgBr₂, TiCl₄, GaCl₂, SnCl₄, FeCl₃, HCl, SiCl₄, BCl₃, TeCl₄, C₆F₅CO₂H, GeI₄, BEt₃.

3. (canceled)

4. The method of claim 1, wherein the catalyst is one or more selected from the group consisting of BF₃-etherate, TFA, or p-CH₃C₆H₄SO₃H·H₂O.

5. The method of claim 1, wherein the catalyst is boron trifluoride etherate.

6. The method of claim 1, wherein the salt is one or more of the salts selected from the group consisting of LiCl, NaCl, KCl, CsCl, MgCl₂, CaCl₂, NH₄Cl, Me₄NCl, NaBPh₄, BaBr, KI, Na₂SO₄, MgSO₄, CaSO₄, NaBF₄, Me₄NBF₄, or KF.

7. The method of claim 1, wherein the salt is one or more of the salts selected from the group consisting of LiCl, NaCl, KCl, CsCl, MgCl₂, CaCl₂, Paraquat 2Cl—, NH₄Cl, Me₄NCl, NaBPh₄, BaBr, or KI.

8. The method of claim 1, wherein the salt is one or more of the salts selected from the group consisting of NaCl, Me₄NCl, or NaBF₄.

9. The method of claim 1, wherein the salt is a metal salt that includes an anion selected from the group consisting of Cl—, Ph₄B—, Br—, or I—.

10. (canceled)

11. The method of claim 9, wherein the salt is sodium chloride and the solvent is a halogenated solvent.

12. (canceled)

13. (canceled)

14. The method of claim 1, wherein the solvent is dichloromethane.

15. (canceled)

16. The method of claim 1, wherein the peroxide is a hydroperoxide.

17. The method of claim 1, wherein the oxidizing agent is hydrogen peroxide or tert-butyl hydroperoxide.

18. The method of claim 1, wherein the pyrrole or substituted pyrrole, the aliphatic or aromatic aldehyde, the salt, and solvent are mixed before the catalyst is added and the porphyrinogen is mixed with the catalyst.

19. (canceled)

20. The method of claim 18, wherein the porphyrinogen is mixed for about 5 minutes or more and about 15 minutes or less.

21. The method of claim 1, wherein the porphyrinogen is mixed with the oxidizing agent for 8 hours or more and 24 hours or less.

22. (canceled)

23. The method of claim 1, further comprising extracting the oxidizing agent with a polar aprotic solvent before being mixed with the porphyrinogen.

24. The method of claim 1, further comprising extracting an aqueous hydrogen peroxide solution with a polar aprotic solvent to remove water and form an anhydrous peroxide solution.

25. The method of claim 24, wherein the solvent is an ether or a diethyl ether.

26. The method of claim 1, the porphyrin comprises tetraphenylporphyrin.

27. The method of claim 1, further comprising a step of contacting the porphyrin with a metalating agent that includes an aluminum compound or a chromium compound.

28. (canceled)
